(19) United States
(12) Patent Application Publication
(10) Pub. No.: US 2026/0219162 A1
(43) Pub. Date: Jul. 30, 2026

(54) FOCUSING METHOD, OPTICAL IMAGING SYSTEM, SEQUENCING SYSTEM, AND MEDIUM

(71) Applicant: GENEMIND BIOSCIENCES COMPANY LIMITED, Shenzhen, Guangdong (CN)

(72) Inventors: Ruitao SUN, Shenzhen (CN); Linsen LI, Shenzhen (CN); Jianfeng XU, Shenzhen (CN); Yongbo ZHAN, Shenzhen (CN)

(73) Assignee: GENEMIND BIOSCIENCES COMPANY LIMITED, Shenzhen, Guangdong (CN)

(21) Appl. No.: 19/145,304

(22) PCT Filed: Jan. 3, 2024
(86) PCT No.: PCT/CN2024/070245

§ 371 Date: Jul. 2, 2025

(30) Foreign Application Priority Data
Jan. 6, 2023 (CN) 202310020929.X
Dec. 11, 2023 (CN) 202311700089.8

(51) Int. Cl. G01N 15/1434; G02B 7/36

(52) U.S. Cl. CPC G01N 15/1434; G02B 7/36; G01N 2015/1452

(57) ABSTRACT

The present application discloses a focusing method. The method comprises: enabling a light source to emit a first light beam to a sample; enabling a driving module to drive an objective lens to move towards the sample at a first step size along an optical axis of the objective lens, and after each movement, acquiring a first position of the objective lens and first light beam information which is received by a focusing sensor and is reflected from the surface of the sample; determining an optical focusing position of the objective lens on a target surface on the basis of the first position and the first light beam information; and on the basis of the optical focusing position, enabling the driving module to drive the objective lens to move at a second step size along the optical axis of the objective lens, and after each movement, acquiring a second position of the objective lens, enabling an image sensor to perform imaging on an object to be tested on the target surface to form a first image, and taking the second position of the objective lens corresponding to the first image having the optimal image quality as a first image focusing position of the objective lens on the target surface. By means of the focusing method, accurate focusing of an objective lens on a sample can be achieved, and a clear image is obtained.

### TECHNICAL FIELD

The present application relates to the field of optical detection, and in particular, to a focusing method, an optical imaging system, a sequencing system, and a medium.

### BACKGROUND

The base sequence determination of nucleic acid samples is usually implemented based on a gene sequencing platform. Currently, the commercially available sequencing platforms include first-generation, second-generation, and third-generation sequencing platforms. From the perspective of functional control, the sequencing platform includes a detection module that is used to convert and/or collect information changes generated by biochemical reactions in sequence determination, thereby enabling base sequence determination. The detection module generally includes an optical detection module, a current detection module, and/or an acid-base (pH) detection module, depending on different sequencing principles. The sequencing platform based on optical detection principles performs sequence determination by analyzing changes in optical signals detected and acquired during sequencing biochemical reactions.

Numerous recent advances in the field of biology have benefited from improved optical imaging systems and technologies, such as those used in sequencing platforms. Maintaining accurate focus during imaging with these optical imaging systems can be critical to the success of imaging operations. Therefore, the focal plane of the system is often calibrated and maintained prior to the use of the system. However, in the actual application, external interferences can easily occur during imaging using an optical imaging system, resulting in focusing or focus tracking failure. For example, when the optical imaging system is used to perform sequence determination, if the object is a nucleic acid molecule in a flowcell, it is easy for the optical imaging system to fail to track the focus due to, for example, bubbles and agglomerates of fluorescent impurities in the liquid inside the flowcell or dust and scratches on the surface of the flowcell. For example, when the optical imaging system is used for imaging, focus tracking of the optical imaging system may fail due to vibration caused by external factors. In the case that the optical imaging system fails to track the focus, if the optical imaging system cannot re-track the focus, the resulting image may be blurred, which makes it impossible to complete the base sequence determination.

### SUMMARY

The present application aims to solve at least one of the above technical problems to at least some extent.

According to a first aspect, provided is a focusing method. The method is applicable to an optical imaging system. The optical imaging system includes a carrying platform and an imaging apparatus, where the carrying platform is configured to carry a sample, and the sample includes a plurality of axially movable surfaces; at least one of the plurality of axially movable surfaces is loaded with an object of interest, and the surface loaded with the object of interest is defined as a target surface for imaging; the imaging apparatus includes a driving module, an objective lens, a focusing module, and an image sensor, and the focusing module includes a light source and a focusing sensor. The method includes:
enabling the light source to emit a first light beam onto the sample;
enabling the driving module to drive the objective lens along an optical axis thereof to move toward the sample at a first step, and acquiring, after each movement, a first position of the objective lens and first light beam information reflected from a surface of the sample and received by the focusing sensor;
identifying, based on the first position and the first light beam information, an optical focusing position of the objective lens for the target surface; and
enabling, based on the optical focusing position, the driving module to drive the objective lens to move along the optical axis thereof at a second step, acquiring, after each movement, a second position of the objective lens and enabling the image sensor to image the object of interest on the target surface to form a first image, and using the second position of the objective lens corresponding to the first image having optimal image quality as a first image focusing position of the objective lens for the target surface.

According to a second aspect, provided is an optical imaging system. The optical imaging system includes:
a carrying platform, configured to carry a sample, where the sample includes a plurality of axially movable surfaces, at least one of the plurality of axially movable surfaces is loaded with an object of interest, and the surface loaded with the object of interest is defined as a target surface for imaging;
an imaging apparatus, including an objective lens, a driving module, a focusing module, and an image sensor, where the focusing module includes a light source and a focusing sensor; and
a controller, configured to:
control the light source to emit a first light beam onto the sample;
control the driving module to drive the objective lens along an optical axis thereof to move toward the sample at a first step, and acquire, after each movement, a first position of the objective lens and first light beam information reflected from a surface of the sample and received by the focusing sensor;
identify, based on the first position and the first light beam information, an optical focusing position of the objective lens for the target surface; and
control, based on the optical focusing position, the driving module to drive the objective lens to move along the optical axis thereof at a second step, acquire, after each movement, a second position of the objective lens and control the image sensor to image the object of interest on the target surface to form a first image, and use the second position of the objective lens corresponding to the first image having optimal image quality as a first image focusing position of the objective lens for the target surface.

According to a third aspect, provided is a sequencing system. The sequencing system includes the optical imaging system described above.

According to a fourth aspect, provided is a computer-readable storage medium. The medium has a program stored thereon, and the program is executable by a processor to implement the method according to the first aspect.

According to the focusing method in the above embodiment, accurate focusing can be achieved to acquire a clear image by: enabling a light source to emit a first light beam onto a sample, using the driving module to drive an objective lens to move along the optical axis thereof toward a sample at a first step, and acquiring, after each movement, a first position of the objective lens and first light beam information reflected from the surface of the sample and received by the focusing sensor; identifying, based on the first position and the first light beam information, an optical focusing position of the objective lens for the target surface; and controlling, based on the optical focusing position, the driving module to drive the objective lens to move along the optical axis thereof at a second step, and acquiring, after each movement, a second position of the objective lens and enabling the image sensor to image the object of interest on the target surface to form a first image, and using the second position of the objective lens corresponding to the first image having the optimal image quality as a first image focusing position of the objective lens for the target surface.

The additional aspects and advantages of the present application will be partially set forth in the following description, and will partially become apparent from the following description or be learned through the practice of the present application.

### BRIEF DESCRIPTION OF THE DRAWINGS

FIG. 1 is a schematic diagram of the structure of an optical imaging system according to one embodiment;

FIG. 2 is a schematic diagram of the structure of an optical imaging system according to another embodiment;

FIG. 3 is a schematic diagram of the structure of a sample (flowcell) according to one embodiment; and

FIG. 4 is a schematic flowchart of a focusing method according to one embodiment.

### DETAILED DESCRIPTION

The present application will be illustrated in further detail with reference to the following detailed description and drawings. Similar elements in different embodiments are denoted by similar reference numerals associated therewith. In the following embodiments, numerous specific details are given to provide a thorough understanding of the present application. However, those skilled in the art will readily recognize that some of the features may be omitted or substituted with other elements, materials, and methods in different instances. In some instances, certain operations related to the present application are not illustrated or described in this specification to avoid obscuring the key part of the present application with unnecessary detail. For those skilled in the art, it is not necessary to describe in detail these related operations, as such operations can be fully understood from the description in the specification and the general knowledge in the art.

Furthermore, the illustrated features, operations, or characteristics in the specification may be combined in any suitable manner to give various embodiments. Also, the various procedures or actions in the description of the methods may be exchanged or adjusted in order, as will be apparent to those skilled in the art. Therefore, the various orders in the specification and drawings are for the purpose of clearly illustrating certain embodiments only and are not intended to imply a necessary order unless otherwise indicated that a certain order is necessary.

The serial numbers used herein for the components, such as “first” and “second” are used merely to distinguish between the objects described, and do not carry any sequential or technical meaning. The terms “connect” and “couple” as used in the present application include both direct and indirect connections (couplings), unless otherwise specified.

In current sequencing platforms, when using an optical imaging system to image an object of interest on a flowcell for base sequence determination (hereinafter referred to as “sequencing”), in order to give clear images, the imaging system needs to perform focusing, for example, by moving the flowcell and/or the objective lens of the optical imaging system, such that the flowcell is positioned on a focal plane of the objective lens. After the focusing succeeds, the process of recording the relative distance between the objective lens and the flowcell when the optical imaging system acquires a clear image and maintaining this relative distance may be referred to as focus locking. When the relative position of the objective lens and the flowcell is changed due to external vibration or other factors, the interference caused by the vibration is eliminated by adjusting to restore the objective lens and the flowcell to their relative distance at the time of focus locking. This process may be referred to as focus tracking. Focus tracking may be conducted manually or automatically by the optical imaging system, and the latter is referred to as automatic focus tracking.

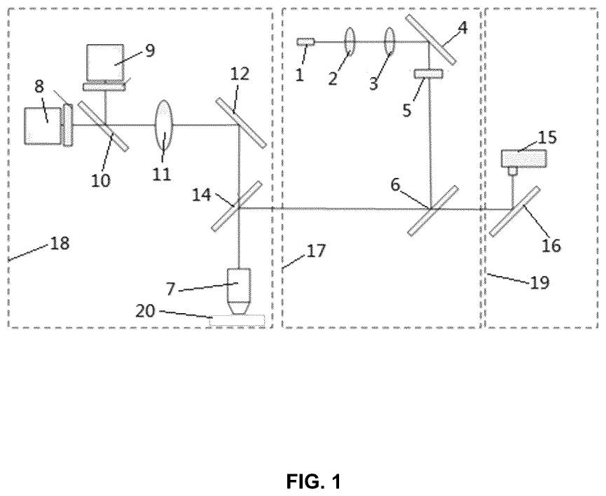

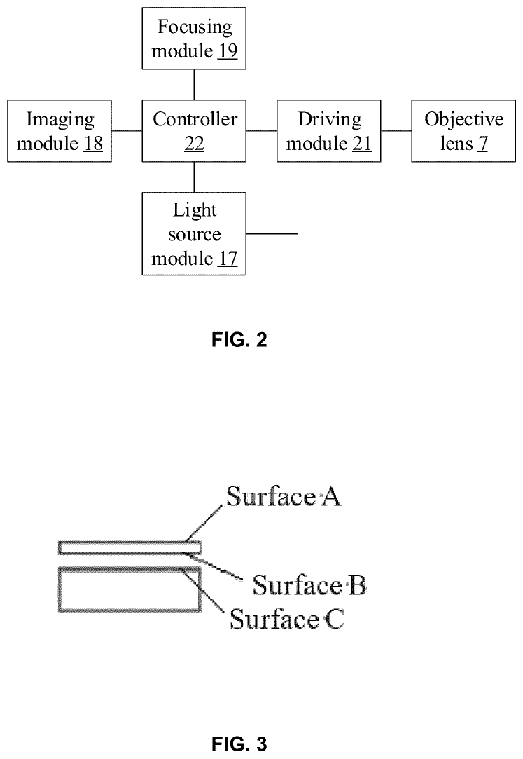

Referring to FIGS. 1 and 2, in some embodiments, provided is an optical imaging system. The optical imaging system includes a carrying platform 20 and an imaging apparatus. The imaging apparatus includes a light source module 17, an imaging module 18, a focusing module 19, a driving module 21, and a controller 22, which will be described in detail below.

The carrying platform 20 is configured to carry a sample. The sample may be a gene sequencing flowcell for carrying an object of interest, e.g., DNA fragments, RNA fragments, and other biomolecules. Referring to FIG. 3, in some embodiments, the sample includes a plurality of surfaces axially movable relative to the optical axis of the objective lens. For example, when the sample includes an upper glass plate and a lower glass plate, as well as a channel (or fluid channel) sandwiched between the upper glass plate and the lower glass plate, the plurality of axially movable surfaces include an upper surface (surface A) of the upper glass plate, a lower surface (surface B) of the upper glass plate, and an upper surface (surface C) of the lower glass plate. At least one of the plurality of axially movable surfaces is loaded with an object of interest, while the surface loaded with the object of interest serves as a target surface for imaging. For example, surface B is specially chemically modified, and the surface thereof is connected with an oligonucleotide sequence via a hydrogen bond, and the oligonucleotide sequence can be complementarily hybridized with an adapter sequence of a single-stranded DNA or RNA. That is, the object of interest is positioned on surface B. Therefore, surface B is the target surface that will eventually undergo the optical focusing and image focusing as described below. In some embodiments, the object of interest is positioned on surface C, and thus surface C is the target surface that will eventually undergo the optical focusing and image focusing as described below. In some embodiments, the object of interest is positioned on surfaces B and C, and thus surfaces B and C are target surfaces that will eventually undergo the optical focusing and image focusing as described below. In some embodiments, the sample may include a plurality of fields of view (FOVs, which refer to the range observable by the objective lens of the optical imaging system at a time). Therefore, the optical imaging system, after completing image acquisition for one FOV of the sample, needs to move to another FOV of the sample, and this process continues until the image acquisition has been completed for all FOVs.

Referring to FIG. 1, the focusing module 19 includes a first light source and a focusing sensor 15. The first light source is configured to provide a first light beam, e.g., infrared light at 850 nm. The objective lens 7 is configured to project the first light beam onto the sample and collect the first light beam reflected from the surface of the sample. The focusing sensor 15 receives the first light beam reflected from the surface of the sample and collected by the objective lens 7 to obtain the first light beam information. In some embodiments, the focusing sensor 15 includes a CCD array therein. The CCD array can detect the shape and size of the spot of the first light beam, and whether the sample is positioned on the focal plane of the objective lens is determined based on the shape and size of the spot, thereby achieving focusing detection. Moreover, the CCD array can also perform photoelectric conversion on the detected first light beam and then output a corresponding voltage. In some embodiments, the first light source may be a built-in light source of the focusing sensor 15. In some embodiments, the focusing module 19 further includes a third reflector 16.

Referring to FIG. 1, the light source module 17 includes a second light source 1, a collimating lens 2, a converging lens 3, a first reflector 4, a first filter 5, and a first dichroic mirror 6. The second light source 1 emits a second light beam. The second light beam is a dual-wavelength excitation light, e.g., lasers at 640 nm and 532 nm. Upon irradiating the object of interest on the sample, the second light beam is used to excite different fluorophores labeled on different bases in the object of interest to generate fluorescence.

Referring to FIG. 1, the imaging module 18 includes an objective lens 7 and an image sensor. The objective lens 7 is configured to, when the second light beam emitted by the second light source 1 passes through the objective lens 7, project the second light beam onto the sample and collect the fluorescence generated by the object of interest on the sample. The image sensor is configured to image the fluorescence collected by the objective lens 7 so as to perform image acquisition on the object of interest. In some embodiments, the imaging module 18 includes a first camera 8 and a second camera 9 equipped with the above image sensor. The imaging module 18 further includes a second dichroic mirror 10, a tube lens 11, a second reflector 12, and a third dichroic mirror 14. A second filter is arranged between the first camera 8 and the second dichroic mirror 10, and a third filter is arranged between the second camera 9 and the second dichroic mirror 10. Specifically, the second light beam emitted by the second light source 1 sequentially passes through the collimating lens 2, the converging lens 3, the first reflector 4, the first filter 5, and the first dichroic mirror 6, and is then reflected by the third dichroic mirror 14 into the objective lens 7; the second light beam is then projected onto the sample by the objective lens 7 to excite different fluorophores on the object of interest to emit fluorescence. The fluorescence is received by the objective lens 7 before sequentially passing through the third dichroic mirror 14, the second reflector 12, and the tube lens 11, and is then divided into a third light beam and a fourth light beam by the second dichroic mirror 10. The third light beam is transmitted by the second dichroic mirror 10 and is received by the first camera 8 after passing through the second filter, and the fourth light beam is reflected by the second dichroic mirror 10 and is received by the second camera 9 after passing through the third filter. The third light beam includes a fluorescence signal at a first wavelength, e.g., a signal representing base A; the fourth light beam includes a fluorescence signal at a second wavelength, e.g., a signal representing base T.

Referring to FIG. 1, in some embodiments, the second light source 1 emits the second light beam; the second light beam sequentially passes through the collimating lens 2 (for collimating and expanding the beam) and the converging lens 3. Then, the second light beam is reflected by the first reflector 4, then transmitted by the first filter 5, and sequentially reflected by the first dichroic mirror 6 (for reflecting laser beams at 640 nm and 532 nm) and the third dichroic mirror 14 (for reflecting laser beams at 640 nm and 532 nm). Finally, the second light beam passes through the objective lens 7 to irradiate the sample and excite the object of interest on the sample to generate fluorescence. The fluorescence collected by the objective lens 7 is transmitted by the third dichroic mirror 14 (for transmitting the fluorescence), then reflected by the second reflector 12 onto the tube lens 11, and converged and emitted by the tube lens 11 to the second dichroic mirror 10. The second dichroic mirror 10 receives the light beam from the tube lens 11 and divides the light beam into a third light beam and a fourth light beam. The third light beam is transmitted by the second dichroic mirror 10 and is received by the first camera 8 after passing through the second filter, and the fourth light beam is reflected by the second dichroic mirror 10 and is received by the second camera 9 after passing through the third filter. The third light beam includes a fluorescence signal at a first wavelength, e.g., a signal representing base A; the fourth light beam includes a fluorescence signal at a second wavelength, e.g., a signal representing base T.

Referring to FIG. 1, in some embodiments, the first light source emits the first light beam; the first light beam is reflected by the third reflector 16, transmitted by the first dichroic mirror 6 (for transmitting infrared light at 850 nm), then reflected by the third dichroic mirror 14 onto the objective lens 7, and finally passes through the objective lens 7 to irradiate the sample. The first light beam reflected from the surface of the sample is collected by the objective lens 7, reflected by the third dichroic mirror 14, transmitted by the first dichroic mirror 6, and finally reflected by the third reflector 16 to the focusing sensor 15.

The driving module 21 is configured to drive the objective lens to move. In some embodiments, the driving module 21 is configured to drive the objective lens to move along the optical axis thereof, e.g., in a vertical direction, such that the distance between the objective lens and the sample can be adjusted. This ensures that the target surface (e.g., surface B) of the sample is positioned on the focal plane of the objective lens, thus completing the optical focusing or image focusing on the sample. In some embodiments, the driving module 21 may drive the objective lens to move using devices such as a servo motor or a step motor or drive the objective lens to move using devices such as a linear module or a lead screw.

In some embodiments, when the driving module 21 drives the objective lens to move such that the target surface (e.g., surface B) of the sample is positioned on the focal plane of the objective lens, it can be said that focusing has been successfully achieved. In some embodiments, whether the surface of the sample is positioned on the focal plane of the objective lens can be determined based on the detection result of the focusing sensor 15. For example, when the surface of the sample is positioned on the focal plane of the objective lens, the amount of light that can be reflected by the surface is the greatest. Therefore, the light intensity value of the reflected light detected by the focusing sensor is also the greatest. In some embodiments, the cross-section of the first light beam provided by the first light source is semicircular, and the first light beam converges to a circular dot on the focal plane after passing through the objective lens. When the surface of the sample is positioned on the focal plane of the objective lens, the spot of the first light beam reflected from the sample surface on the CCD array of the focusing sensor is also a circular dot. When the surface of the sample is positioned above the focal plane of the objective lens, the spot of the reflected light on the CCD array of the focusing sensor appears as a right semicircle; when the surface of the sample is positioned below the focal plane of the objective lens, the spot of the reflected light on the CCD array of the focusing sensor appears as a left semicircle. Therefore, whether the surface of the sample is positioned on the focal plane of the objective lens, i.e., whether the focusing succeeds, can be determined by the light intensity value and spot information of the first light beam reflected from the surface of the sample and detected by the focusing sensor 15. The process of determining whether the focusing succeeds based on the detection result of the focusing sensor 15 may be referred to as the optical focusing process. The first light beam information includes a light intensity value and/or spot information of the first light beam.

In some embodiments, whether the target surface (e.g., surface B) of the sample is positioned on the focal plane of the objective lens can also be determined based on the definition of the image acquired by the image sensor. For example, when the target surface of the sample is positioned on the focal plane of the objective lens, it represents the optimal position for the objective lens to collect the fluorescence signal. Therefore, the image definition obtained by the image sensor after imaging the object of interest is also optimal, such as achieving the highest definition. When the target surface of the sample is positioned above or below the focal plane of the objective lens, the image definition obtained by the image sensor after imaging the object of interest is lower than the optimal definition. Therefore, by assessing the definition of the image, it is possible to determine whether the target surface of the sample is positioned on the focal plane of the objective lens, i.e., whether the focusing succeeds. The process of determining whether the focusing succeeds based on the image acquired by the image sensor may be referred to as the image focusing process.

In some embodiments, since the fluorescence generated by the object of interest is finally imaged by the image sensor to achieve the sequencing of the base sequence of the object of interest, successful image focusing is ultimately required. In some embodiments, the definition of the image obtained by the image sensor imaging the object of interest on the target surface after image focusing is greater than the definition of the image obtained by the image sensor imaging the object of interest on the target surface after optical focusing. Therefore, optical focusing is required first to locate the target surface (e.g., surface B) of the sample based on the detection result of the focusing sensor 15, and the position of the objective lens corresponding to the target surface is acquired when the optical focusing succeeds. The optical focusing can be regarded as coarse focusing. That is, the position of the objective lens corresponding to the target surface acquired based on optical focusing is not yet the optimal position for optical imaging and cannot be directly used for final optical exposure and image acquisition. Instead, based on the position of the objective lens corresponding to the target surface acquired by optical focusing, fine focusing of the target surface of the sample via image focusing is needed; then the position of the objective lens at which the image sensor achieves the optimal image definition after imaging the target surface is identified, and this position is used for final optical exposure and image acquisition. In this embodiment, the objective lens is used to perform optical focusing on the sample to locate the target surface, and then image focusing is performed on the target surface, such that the objective lens can accurately focus on the sample, thereby improving the definition of the image.

The controller 22 is configured to control, based on the current position of the objective lens 7, the driving module 21 to drive the objective lens 7 to move and, after each movement, control the focusing sensor 15 to perform detection to perform optical focusing on the surface of the sample. Moreover, when the optical focusing succeeds, the position of the objective lens is acquired and used as the optical focusing position.

In some embodiments, in the initial state, the current position of the objective lens may be an approximate position adjusted manually, and then optical focusing is performed based on the current position of the objective lens. In some embodiments, the current position of the objective lens may also be the focusing position of the previous sample, and then optical focusing is performed based on the current position of the objective lens after the previous sample is replaced by the current sample.

In some embodiments, when the sample has a plurality of surfaces, only one of the surfaces is the target surface. Therefore, the target surface needs to be found first. In some embodiments, based on the current position of the objective lens, the driving module is controlled to drive the objective lens to move along the optical axis thereof within a first range at a first step. For example, if the current position of the objective lens is at coordinate Z=Z1 in the direction of the optical axis, then the first range may be (Z1+X, Z1−X), and the controller 22 controls the driving module to drive the objective lens to scan from coordinate Z1−X to coordinate Z1+X in the direction of the optical axis at the first step. In some embodiments, X is set to a value of 175 μm and the first step is set to a value of 2.0 μm, such that the surface of the sample can be sought in a larger range. After each movement of the objective lens, the controller 22 controls the focusing sensor 15 to detect the first light beam reflected from the surface of the sample. After the objective lens completes the scan within the first range, the controller 22 determines whether the surface is detected based on the detection result. In some embodiments, the relative distance between the focal plane of the objective lens and the surface of the sample in each detection can be obtained based on the light intensity value and the spot information of the first light beam reflected from the surface of the sample in each detection. For example, when the light intensity value reaches its maximum and the size and shape of the spot is a circular dot, the relative distance between the focal plane of the objective lens and the surface of the sample can be determined as zero or substantially zero (within a tolerance range). Therefore, for each instance where it is detected that the relative distance between the focal plane of the objective lens and the surface of the sample is zero, it can be determined that the surface is detected in this detection. If at least one surface is detected and a target surface is among the at least one surface, the optical focusing succeeds. Moreover, the position of the objective lens when the target surface is detected is acquired and used as the optical focusing position; otherwise, the optical focusing fails.

In some embodiments, when at least one surface is detected and the target surface is among the at least one surface, the controller 22 is configured to respectively acquire the position of the objective lens when each surface is detected, and then obtain the distances between the respective surfaces based on the distances between the respective positions of the objective lens. Then, the positions of the respective surfaces on the sample are determined based on the distances between the respective surfaces. Finally, the target surface is identified from two or more surfaces based on the positions of the respective surfaces on the sample. In this embodiment, the distances between the respective surfaces can be approximately obtained based on the distances between the respective positions of the objective lens. The distances between the respective surfaces are fixed for each type of sample, and thus the positions of the respective surfaces on the sample are determined based on the distances between the respective surfaces. Finally, the target surface is identified based on the positions of a plurality of surfaces on the sample. In some embodiments, the surfaces of the sample are the upper surface of the upper glass plate (surface A), the lower surface of the upper glass plate (surface B), and the upper surface of the lower glass plate (surface C). The thickness of the upper glass plate is 175 μm, the thickness of the fluid channel is 75 μm, and the thickness of the lower glass plate is 500 μm. Therefore, surfaces A, B, and C can be identified based on the distances between the respective surfaces, and the target surface, surface B, is selected. In some embodiments, after obtaining the distances between the respective positions of the objective lens, it is also necessary to take into account that the distance was detected in an air medium to determine the thickness within the glass medium. Therefore, a refractive index conversion is required. That is, the distances between the respective positions of the objective lens are multiplied by the refractive index of the glass to obtain the distances between the respective surfaces.

In some embodiments, the image focusing includes a first image focusing and a second image focusing.

Based on the optical focusing position of the objective lens, the controller 22 controls the driving module to drive the objective lens to move and, after each movement, controls the image sensor to perform image acquisition on the object of interest on the target surface, so as to perform the first image focusing on the object of interest. Moreover, when the first image focusing succeeds, the position of the objective lens is acquired and used as the first image focusing position. Specifically, based on the optical focusing position of the objective lens, the controller 22 controls the driving module to drive the objective lens to move within a second range at a second step. For example, if the optical focusing position of the objective lens is at coordinate Z=Z2 along the optical axis, then the second range may be (Z2+Y, Z2−Y), and the controller 22 controls the driving module 21 to drive the objective lens 7 to stepwise scan from coordinate Z2−Y to coordinate Z2+Y in the direction of the optical axis at the second step. In some embodiments, Y is set to a value of 5 μm, and the second step is set to a value of 1.0 μm, such that the surface can be sought in a smaller range. As can be seen from the second range and the second step, the accuracy of image focusing is higher than that of optical focusing. After each movement of the objective lens, the controller 22 controls the image sensor to perform image acquisition on the object of interest to obtain a first image and acquire a first image having the optimal definition among the first images. If the definition of the first image having the optimal definition is superior to a threshold definition, the image focusing succeeds, and the position of the objective lens while obtaining the first image having the optimal definition is acquired and used as the image focusing position. In this embodiment, the definition of the image can be obtained by evaluating the image. For example, the sharpness value of the image can be calculated and taken as the definition of the image. For example, the bright spots in the images may be evaluated to obtain corresponding scores, and the scores are used as the definition of the images. The score of the bright spots in each image is calculated by the following formula:

$$Score = ( ( k 1 \times k 2 - 1 ) CV - E V ) / ( ( C V + E V ) / ( k 1 \times k 2 ) )$$

The matrix corresponding to the bright spot is defined as a matrix k1×k2 composed of odd rows and odd columns, which contains k1×k2 pixels. CV denotes the central pixel value of the matrix corresponding to the bright spot, and EV denotes the sum of the non-central pixel values of the matrix corresponding to the bright spot. It can be understood that the image definition can also be obtained by evaluating the image in other ways. In some embodiments, once the position of the objective lens for obtaining the first image having the optimal definition is acquired, it is indicated that the position of the objective lens is closest to the optimal focusing distance in the second range. Therefore, when the definition of the first image having the optimal definition is superior to the threshold definition, the image focusing is deemed successful, and the image focusing position of the objective lens is thus acquired. The threshold definition may be an image definition required by the image to satisfy the gene sequencing.

In some embodiments, when the driving module 21 is controlled to drive the objective lens 7 to move within the second range at the second step, the controller 22 is further configured to determine whether the definition of the first image obtained by the image sensor is superior to a preset definition after each movement of the objective lens 7. If the definition is smaller than the preset definition, the second step is maintained, and the driving module 21 is controlled to drive the objective lens 7 to continue moving. If the definition is greater than the preset definition, the second step is reduced, and then the driving module 21 is controlled to drive the objective lens 7 to continue moving. In this embodiment, when the definition of the obtained first image is superior to the preset definition, it indicates that, in this case, the position of the objective lens is getting closer to the optimal focusing distance. Therefore, in order to obtain a more accurate position of the objective lens, the second step should be reduced; otherwise, the second step is maintained to continue approaching the optimal focusing distance. For example, in the case that Y is set to a value of 5 μm and the second step is set to a value of 1.0 μm within the second range of (Z2+Y, Z2−Y), when the definition of the obtained first image is superior to the preset definition, the second step is reduced to 0.3 μm; otherwise, the second step is maintained at 1.0 μm.

The controller 22 is further configured to control, based on the first image focusing position of the objective lens 7, the driving module 21 to drive the objective lens 7 to move and, after each movement, control the image sensor to perform image acquisition on the object of interest to perform the second image focusing on the object of interest. Moreover, when the second image focusing succeeds, the voltage generated by photoelectric conversion of the focusing sensor 15 based on the received first light beam reflected from the target surface is acquired and used as the focus tracking voltage.

In some embodiments, one end of the object of interest having a certain length (e.g., a single-stranded nucleic acid molecule) is fixed on the target surface, and the other end of the object of interest is spaced apart from the target surface to which the object of interest is fixed by a certain distance (e.g., ten to hundred nanometers), such that when the target surface is positioned on the focal plane of the objective lens 7, the object of interest is not actually positioned on the focal plane of the objective lens 7. If, in this case, the voltage generated by photoelectric conversion of the focusing sensor 15 based on the received first light beam reflected from the target surface is used as the focus tracking voltage, a certain degree of deviation may be present, and a longer length of the object of interest leads to a greater deviation. To reduce the deviation, the controller 22 controls the reset of the voltage output by the focusing sensor 15 to the driving module 21, for example, by performing a “make 0” operation to prompt the focusing sensor 15 that it is not the optimal timing to acquire a clear image of the object of interest when the target surface is positioned on the focal plane of the objective lens 7 or when the spot formed on the CCD array of the focusing sensor 15 by the first light beam reflected from the target surface is also a circular dot. In this case, a second image focusing is required to obtain a clear image of the object of interest. Specifically, based on the first image focusing position of the objective lens 7, the objective lens 7 is moved within a third range at a third step. In some embodiments, the third step is equal to the second step. After each movement of the objective lens 7, the image sensor is controlled to perform image acquisition on the object of interest to obtain the second image, and a second image having the optimal definition among the second images is acquired. If the definition of the second image having the optimal definition is superior to the threshold definition, the second image focusing succeeds. In some embodiments, the process of the second image focusing is substantially identical to the process of the first image focusing. For example, if the image focusing position of the objective lens 7 is at coordinate Z=Z3 in the direction of the optical axis, then the third range may be (Z3+Y, Z3−Y), and the controller 22 controls the driving module 21 to drive the objective lens 7 to stepwise scan from coordinate Z3−Y to coordinate Z3+Y in the direction of the optical axis at the third step, where Y is set to a value of 5 μm, and the third step is set to a value of 0.3 μm.

In some embodiments, during the second image focusing, the focusing module 19 also operates. That is, after each movement of the objective lens 7, the focusing module 19 also performs photoelectric conversion on the received first light beam reflected from the target surface and generates a corresponding voltage. Since the first light beam reflected by the target surface is substantially maintained stable when the target surface of the sample is substantially positioned on the focal plane of the objective lens, the voltage generated by the photoelectric conversion is also maintained stable. Therefore, the change in the distance between the focal plane of the objective lens and the target surface of the sample can be determined by the change in the voltage. Therefore, when the second image focusing succeeds, the voltage generated by the photoelectric conversion of the focusing sensor while obtaining the third image having the optimal definition is acquired and used as the focus tracking voltage, thereby ensuring the effectiveness and accuracy of the subsequent focus tracking processes.

In some embodiments, the controller 22 is configured to control the driving module 21 to drive the objective lens 7 to move to the second image focusing position under the focus tracking voltage and to control the image sensor to image the objects of interest at a plurality of positions on the target surface. In the process of imaging the objects of interest at each position by the image sensor, the difference between the voltage generated by the focusing sensor based on the received first light beam reflected from the target surface and the focus tracking voltage is acquired. The controller controls the driving module 21 to drive the objective lens 7 to move based on the difference to eliminate the difference. Specifically, when the objective lens 7 continues to return to its second image focusing position, the controller 22 can control the image sensor to perform image acquisition for objects of interest at different positions, thereby achieving gene sequencing. During image acquisition, various external factors may cause the target surface of the sample to deviate from the focal plane of the objective lens, resulting in changes to the first light beam reflected by the target surface. This, in turn, changes the voltage generated when the focusing sensor 15 performs photoelectric conversion based on the received first light beam reflected from the target surface. For example, such a change may occur when the sample transitions from one FOV to another FOV. Therefore, it is necessary to acquire the difference between the voltage generated by the photoelectric conversion of the focusing sensor 15 based on the received first light beam reflected from the target surface and the focus tracking voltage, and then control the driving module 21 to drive the objective lens 7 to move based on the difference to eliminate the difference, thereby re-positioning the target surface of the sample on the focal plane of the objective lens 7. This process is the automatic focusing process.

In the embodiments of the present application, the first image focusing position of the objective lens is identified by means of the optical focusing and the first image focusing to achieve accurate focusing of the objective lens for the target surface, and then a second image focusing is performed based on the first image focusing position of the objective lens to obtain the focus tracking voltage. Thus, during the gene sequencing, the position of the objective lens can be adjusted based on the difference between the voltage generated by the photoelectric conversion of the focusing sensor based on the received first light beam reflected from the target surface and the focus tracking voltage, so as to eliminate the difference and achieve focus tracking of the objective lens for the target surface, thereby achieving automatic focusing by focusing and focus tracking.

In some embodiments, the focusing method further includes a step of verifying the focus tracking voltage.

Specifically, based on the second image focusing position, the driving module 21 drives the objective lens 7 to move along the optical axis thereof within a fourth range at a fourth step under the focus tracking voltage. After each movement, the image sensor images the object of interest on the target surface to form a third image, and the third image having the optimal image quality is compared with the second image having the optimal image quality. If the image qualities of the third image and the second image are substantially identical, it is determined that the focus tracking voltage passes verification. In some embodiments, the fourth range is identical to the third range, and the fourth step is identical to the third step.

More specifically, when controlling the image sensor to perform image acquisition on the object of interest, the controller 22 is further configured to control the objective lens 7 to be at the second image focusing position, control the focusing sensor 15 to perform photoelectric conversion based on the received first light beam reflected from the target surface to obtain the voltage for verification, then control the driving module 21 to drive the objective lens 7 to move based on the difference between the voltage for verification and the focus tracking voltage, and control the image sensor to perform image acquisition on the object of interest. In this embodiment, before the gene sequencing on the sample is performed, whether the automatic focusing is correct needs to be further verified. Therefore, based on the second image focusing position, the focusing sensor 15 is controlled to perform photoelectric conversion based on the received first light beam reflected from the target surface to obtain the voltage for verification. Then, the objective lens is controlled to move based on the difference between the voltage for verification and the focus tracking voltage, and then the image acquisition is performed on the object of interest once to obtain the image for verification. Then, the image acquired by the image sensor of the object of interest when the second image focusing succeeds is acquired and used as the reference image. It is determined whether there is a change in definition between the verified image and the reference image. If the definitions of the verified image and the reference image are substantially identical, it indicates that the verification is passed. Thus, the obtained focus tracking voltage can be used for automatic focusing and to control the image sensor to perform image acquisition on the object of interest.

In some embodiments, if the optical focusing fails, the image focusing includes a third image focusing. That is, based on the current position of the objective lens, the driving module 21 is controlled to drive the objective lens 7 to move, and after each movement, the image sensor is controlled to perform image acquisition to perform the third image focusing on the object of interest. Moreover, when the third image focusing succeeds, a fourth position of the objective lens is acquired and used as the optical focusing position. In this embodiment, when optical focusing fails due to reasons of the device or the sample, for example, positional deviation of the optical device (the focusing module) or temperature change of the sample, the focusing module 19 fails to detect the surface of the sample in its detection results. In this case, to avoid the focusing failure of the current sample, a third image focusing can be performed by the image sensor to locate the surface of the sample.

In some embodiments, the process of the third image focusing is substantially identical to the process of the first image focusing, except for the moving range and the moving step of the objective lens. That is, the moving range and the moving step of the objective lens during the third image focusing are both greater than those during the first image focusing. In some embodiments, the controller 22 is configured to control the driving module 21 to drive the objective lens 7 to move within a fifth range at a fifth step at the current position of the objective lens 7. In some embodiments, the moving range and the moving step of the objective lens 7 during the third image focusing are identical to those during the optical focusing. That is, the fifth range may also be (Z1+X, Z1−X), where X is also set to a value of 175 μm and the fifth step is also set to a value of 2.0 μm, such that the surface can be sought in a larger range. After each movement of the objective lens 7, the controller 22 controls the image sensor to perform image acquisition on the object of interest to obtain a fourth image and to determine whether the surface is detected based on the change in the definition of each fourth image. When the surface is positioned on the focal plane of the objective lens, the definition of the image acquired by the image sensor is greater than the definition of the images acquired when the surface is positioned above or below the focal plane of the objective lens. Therefore, the surface can be located based on the change in the definition of each fourth image. In some embodiments, when determining whether the surface is detected based on the change in the definition of each fourth image, the controller 22 is configured to sequentially acquire the definition of each fourth image according to the moving sequence of the objective lens. When the definition of one fourth image is superior to the definition of the two adjacent fourth images thereof, it is indicated that the definition of the images is increased and then decreased. In this case, it can be determined that the surface is detected when the fourth image is obtained. If at least one surface is detected and the target surface is among the at least one surface, the third image focusing succeeds. Moreover, the fourth position of the objective lens when the target surface is detected is acquired and used as the optical focusing position. In this embodiment, the image sensor 15 is used to perform the third image focusing on the sample, such that the optical focusing can be remedied when the focusing module 19 fails to focus.

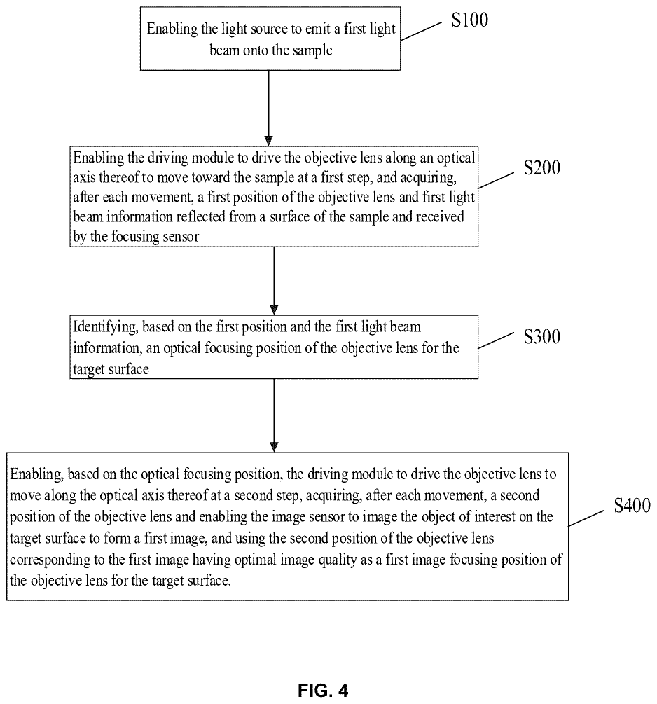

Referring to FIG. 4, in some embodiments, provided is a focusing method. The method is applicable to an optical imaging system. The optical imaging system includes a carrying platform and an imaging apparatus. The carrying platform is configured to carry a sample, and the sample includes a plurality of axially movable surfaces. At least one of the plurality of axially movable surfaces is loaded with an object of interest, and the surface loaded with the object of interest is defined as a target surface for imaging. The imaging apparatus includes a driving module, a focusing module, an objective lens, and an image sensor; the focusing module includes a light source and a focusing sensor. The method includes:

step S100: enabling the light source to emit a first light beam onto the sample;

step S200: enabling the driving module to drive the objective lens along the optical axis thereof to move toward the sample at a first step and, after each movement, acquiring a first position of the objective lens and first light beam information reflected from the surface of the sample and received by the focusing sensor;

step S300: identifying, based on the first position and the first light beam information, an optical focusing position of the objective lens for the target surface; and

step S400: enabling, based on the optical focusing position, the driving module to drive the objective lens to move along the optical axis thereof at a second step, acquiring, after each movement, a second position of the objective lens and enabling the image sensor to image the object of interest on the target surface to form a first image, and using a second position of the objective lens corresponding to the first image having the optimal image quality as a first image focusing position of the objective lens for the target surface.

In some embodiments, the first step is greater than the second step.

In some embodiments, the method includes: enabling, based on the first image focusing position, the driving module to drive the objective lens to move along the optical axis thereof at a third step; acquiring, after each movement, a third position of the objective lens and enabling the image sensor to image the object of interest on the target surface to form a second image; and using the third position of the objective lens corresponding to the second image having the optimal image quality as a second image focusing position of the objective lens for the target surface.

The voltage generated by the focusing sensor based on the received first light beam reflected from the target surface when the objective lens is positioned at the second image focusing position is acquired and used as the focus tracking voltage.

In some embodiments, the method includes: enabling, based on the second image focusing position, the driving module to drive the objective lens to move along the optical axis thereof at a fourth step under the focus tracking voltage; enabling, after each movement, the image sensor to image the object of interest on the target surface to form a third image; and comparing the third image having the optimal image quality with the second image having the optimal image quality, where if the image qualities of the third image and the second image are substantially identical, it is determined that the focus tracking voltage passes verification.

In some embodiments, the method includes: enabling the driving module to drive the objective lens to move to the second image focusing position under the focus tracking voltage; imaging the objects of interest at a plurality of positions on the target surface using the image sensor; acquiring a difference between a voltage generated by the focusing sensor based on the received first light beam reflected from the target surface and the focus tracking voltage in the process of imaging the objects of interest at each position by the image sensor; and controlling the driving module to drive the objective lens to move based on the difference to eliminate the difference.

In some embodiments, identifying, based on the first position and the first light beam information, the optical focusing position of the objective lens for the target surface includes:
determining, based on the first light beam information, whether at least one surface of the sample is detected;
determining, if at least one surface of the sample is detected, whether the target surface exists among the at least one surface of the sample that has been detected; and
acquiring, if the target surface is detected among the at least one surface of the sample, a first position of the objective lens when the target surface is detected, and using the first position as the optical focusing position of the objective lens for the target surface.

In some embodiments, determining, based on the first light beam information, whether the at least one surface of the sample is detected includes:
obtaining, based on the first light beam information, a relative distance between a focal plane of the objective lens and a surface of the sample; and for each instance where the relative distance between the focal plane of the objective lens and the surface of the sample is zero, determining that one surface of the sample is detected.

In some embodiments, determining, if at least one surface of the sample is detected, whether the target surface exists among the at least one surface of the sample that has been detected; and acquiring, if the target surface is detected among the at least one surface of the sample, a first position of the objective lens when the target surface is detected, and using the first position as the optical focusing position of the objective lens for the target surface include:
acquiring, respectively, when two or more surfaces of the sample are detected, the first position of the objective lens when each of the surfaces is detected;
obtaining, based on distances between the respective first positions of the objective lens, distances between the respective surfaces;
determining, based on the distances between the respective surfaces, positions of the respective surfaces on the sample;
identifying, based on the positions of the respective surfaces on the sample, the target surface; and
using the first position of the objective lens when the target surface is detected as the optical focusing position of the objective lens for the target surface.

In some embodiments, identifying, based on the first position and the first light beam information, the optical focusing position of the objective lens for the target surface includes:
determining, based on the first position and the first light beam information, whether at least one surface of the sample is detected;
enabling, if the at least one surface of the sample is not detected, the driving module to drive the objective lens to move along the optical axis thereof at a fifth step based on the current position of the objective lens, and acquiring, after each movement, a fourth position of the objective lens and controlling the image sensor to image the sample to form a fourth image;
determining again, based on the image quality change of each fourth image, whether at least one surface of the sample is detected;
determining, if at least one surface of the sample is detected, whether the target surface exists among the at least one surface of the sample that has been detected; and
acquiring, if the target surface is detected among the at least one surface of the sample, a fourth position of the objective lens when the target surface is detected, and using the fourth position as the optical focusing position of the objective lens for the target surface.

In some embodiments, determining, based on the first light beam information, whether the at least one surface of the sample is detected includes:
obtaining, based on the first light beam information, a relative distance between a focal plane of the objective lens and a surface of the sample; and for each instance where the relative distance between the focal plane of the objective lens and the surface of the sample is zero, determining that one surface of the sample is detected.

In some embodiments, determining again, based on the image quality change of the each fourth image, whether the surface of the sample is detected includes:
acquiring the each fourth image sequentially according to a moving sequence of the objective lens; and
determining, for each instance where the image quality of a fourth image is superior to the image quality of the two adjacent fourth images thereof, that one surface of the sample is detected when the fourth image is obtained.

In some embodiments, determining, if at least one surface of the sample is detected, whether the target surface exists among the at least one surface of the sample that has been detected; and acquiring, if the target surface is detected among the at least one surface of the sample, a fourth position of the objective lens when the target surface is detected, and using the fourth position as the optical focusing position of the objective lens for the target surface
include:
acquiring, respectively, when two or more surfaces of the sample are detected, the fourth position of the objective lens when each of the surfaces is detected;
obtaining, based on distances between the respective fourth positions of the objective lens, distances between the respective surfaces;
determining, based on the distances between the respective surfaces, positions of the respective surfaces on the sample;
identifying, based on the positions of the respective surfaces on the sample, the target surface; and
using the fourth position of the objective lens when the target surface is detected as the optical focusing position of the objective lens for the target surface.

In some embodiments, the first light beam information includes a light intensity value and/or spot information of the first light beam.

In some embodiments, provided is a sequencing system. The sequencing system includes the optical imaging system described above.

In some embodiments, provided is a computer-readable storage medium. The medium has a program stored thereon, and the program is executable by a processor to implement the focusing method described above.

Those skilled in the art can understand that all or part of the functions of the methods in the above embodiments may be implemented by hardware or by a computer program. When all or part of the functions of the above embodiments are implemented by a computer program, the program may be stored on a computer-readable storage medium, and the storage medium may include: a read-only memory, a random access memory, a magnetic disk, an optical disk, a hard disk, etc. The program is executed by a computer to implement the above functions. For example, the program may be stored on a memory of a device, and when the program in the memory is executed by a processor, all or part of the functions described above can be implemented. In addition, when all or part of the functions in the above embodiments are implemented by a computer program, the program may be stored on a storage medium such as a server, another computer, a magnetic disk, an optical disk, a flash disk, or a portable hard disk, may be downloaded, copied or saved on a memory of a local device, or may update a system of the local device; when the program on the memory is executed by a processor, all or part of the functions in the above embodiments may be implemented.

The present application has been illustrated by means of specific examples, which, however, are provided to help understand the present application, rather than limiting the present application. Numerous simple deductions, modifications, or substitutions may also be made by those skilled in the art to which the present application belongs in light of the present teachings.

1. A focusing method, applicable to an optical imaging system, wherein the optical imaging system comprises a carrying platform and an imaging apparatus, wherein the carrying platform is configured to carry a sample, and the sample comprises a plurality of axially movable surfaces; at least one of the plurality of axially movable surfaces is loaded with an object of interest, and the surface loaded with the object of interest is defined as a target surface for imaging; the imaging apparatus comprises a driving module, a focusing module, an objective lens, and an image sensor, and the focusing module comprises a light source and a focusing sensor; and wherein the method comprises:
enabling the light source to emit a first light beam onto the sample;
enabling the driving module to drive the objective lens along an optical axis thereof to move toward the sample at a first step, and acquiring, after each movement, a first position of the objective lens and first light beam information reflected from a surface of the sample and received by the focusing sensor;
identifying, based on the first position and the first light beam information, an optical focusing position of the objective lens for the target surface; and
enabling, based on the optical focusing position, the driving module to drive the objective lens to move along the optical axis thereof at a second step, acquiring, after each movement, a second position of the objective lens and enabling the image sensor to image the object of interest on the target surface to form a first image, and using the second position of the objective lens corresponding to the first image having optimal image quality as a first image focusing position of the objective lens for the target surface.

2. The focusing method according to claim 1, wherein the first step is greater than the second step.

3. The focusing method according to claim 1, comprising: enabling, based on the first image focusing position, the driving module to drive the objective lens to move along the optical axis thereof at a third step, acquiring, after each movement, a third position of the objective lens and enabling the image sensor to image the object of interest on the target surface to form a second image, and using the third position of the objective lens corresponding to the second image having the optimal image quality as a second image focusing position of the objective lens for the target surface; and
acquiring a voltage generated by the focusing sensor based on the received first light beam reflected from the target surface when the objective lens is positioned at the second image focusing position and using the voltage as a focus tracking voltage.

4. The focusing method according to claim 3, comprising:
enabling, based on the second image focusing position, the driving module to drive the objective lens to move along the optical axis thereof at a fourth step under the focus tracking voltage; enabling, after each movement, the image sensor to image the object of interest on the target surface to form a third image; and comparing the third image having the optimal image quality with the second image having the optimal image quality, wherein if the image qualities of the third image and the second image are substantially identical, it is determined that the focus tracking voltage passes verification.

5. The focusing method according to claim 3, comprising: enabling the driving module to drive the objective lens to move to the second image focusing position under the focus tracking voltage; imaging the objects of interest at a plurality of positions on the target surface using the image sensor; acquiring a difference between the voltage generated by the focusing sensor based on the received first light beam reflected from the target surface and the focus tracking voltage in the process of imaging the objects of interest at each position by the image sensor; and controlling the driving module to drive the objective lens to move based on the difference to eliminate the difference.

6. The focusing method according to claim 1, wherein identifying, based on the first position and the first light beam information, the optical focusing position of the objective lens for the target surface comprises:
determining, based on the first light beam information, whether at least one surface of the sample is detected;
determining, if at least one surface of the sample is detected, whether the target surface exists among the at least one surface of the sample that has been detected; and
acquiring, if the target surface is detected among the at least one surface of the sample, a first position of the objective lens when the target surface is detected, and using the first position as the optical focusing position of the objective lens for the target surface.

7. The focusing method according to claim 6, wherein determining,
based on the first light beam information, whether at least one surface of the sample is detected comprises:
obtaining, based on the first light beam information, a relative distance between a focal plane of the objective lens and a surface of the sample; and for each instance where the relative distance between the focal plane of the objective lens and the surface of the sample is zero, determining that one surface of the sample is detected.

8. The focusing method according to claim 6, wherein determining, if at least one surface of the sample is detected, whether the target surface exists among the at least one surface of the sample that has been detected; and acquiring, if the target surface is detected among the at least one surface of the sample, the first position of the objective lens when the target surface is detected, and using the first position as the optical focusing position of the objective lens for the target surface comprise:
acquiring, respectively, when two or more surfaces of the sample are detected, the first position of the objective lens when each of the surfaces is detected;
obtaining, based on distances between the respective first positions of the objective lens, distances between the respective surfaces;
determining, based on the distances between the respective surfaces, positions of the respective surfaces on the sample;
identifying, based on the positions of the respective surfaces on the sample, the target surface; and
using the first position of the objective lens when the target surface is detected as the optical focusing position of the objective lens for the target surface.

9. The focusing method according to claim 1, wherein identifying, based on the first position and the first light beam information, the optical focusing position of the objective lens for the target surface comprises:
determining, based on the first position and the first light beam information, whether at least one surface of the sample is detected;
enabling, if the at least one surface of the sample is not detected, the driving module to drive the objective lens to move along the optical axis thereof at a fifth step based on a current position of the objective lens, and acquiring, after each movement, a fourth position of the objective lens and controlling the image sensor to image the sample to form a fourth image;
determining again, based on an image quality change of each fourth image, whether at least one surface of the sample is detected;
determining, if at least one surface of the sample is detected, whether the target surface exists among the at least one surface of the sample that has been detected; and
acquiring, if the target surface is detected among the at least one surface of the sample, a fourth position of the objective lens when the target surface is detected, and using the fourth position as the optical focusing position of the objective lens for the target surface.

10. The focusing method according to claim 9, wherein determining, based on the first light beam information, whether at least one surface of the sample is detected comprises:
obtaining, based on the first light beam information, a relative distance between a focal plane of the objective lens and a surface of the sample; and for each instance where the relative distance between the focal plane of the objective lens and the surface of the sample is zero, determining that one surface of the sample is detected.

11. The focusing method according to claim 9, wherein determining again, based on the image quality change of the each fourth image, whether the surface of the sample is detected comprises:
acquiring the each fourth image sequentially according to a moving sequence of the objective lens; and
determining, for each instance where the image quality of a fourth image is superior to the image quality of two adjacent fourth images thereof, that one surface of the sample is detected when the fourth image is obtained.

12. The focusing method according to claim 9, wherein determining, if at least one surface of the sample is detected, whether the target surface exists among the at least one surface of the sample that has been detected; and acquiring, if the target surface is detected among the at least one surface of the sample, the fourth position of the objective lens when the target surface is detected, and using the fourth position as the optical focusing position of the objective lens for the target surface comprise:
acquiring, respectively, when two or more surfaces of the sample are detected, the fourth position of the objective lens when each of the surfaces is detected;
obtaining, based on distances between the respective fourth positions of the objective lens, distances between the respective surfaces;
determining, based on the distances between the respective surfaces, positions of the respective surfaces on the sample;
identifying, based on the positions of the respective surfaces on the sample, the target surface; and
using the fourth position of the objective lens when the target surface is detected as the optical focusing position of the objective lens for the target surface.

13. The focusing method according to claim 1, wherein the first light beam information comprises a light intensity value and/or spot information of the first light beam.

14. An optical imaging system, comprising:
a carrying platform, configured to carry a sample, wherein the sample comprises a plurality of axially movable surfaces, at least one of the plurality of axially movable surfaces is loaded with an object of interest, and the surface loaded with the object of interest is defined as a target surface for imaging;
an imaging apparatus, comprising a driving module, a focusing module, an objective lens, and an image sensor, wherein the focusing module comprises a light source and a focusing sensor; and
a controller, configured to:
control the light source to emit a first light beam onto the sample;
control the driving module to drive the objective lens along an optical axis thereof to move toward the sample at a first step, and acquire, after each movement, a first position of the objective lens and first light beam information reflected from a surface of the sample and received by the focusing sensor;
identify, based on the first position and the first light beam information, an optical focusing position of the objective lens for the target surface; and
control, based on the optical focusing position, the driving module to drive the objective lens to move along the optical axis thereof at a second step, acquire, after each movement, a second position of the objective lens and control the image sensor to image the object of interest on the target surface to form a first image, and use the second position of the objective lens corresponding to the first image having optimal image quality as a first image focusing position of the objective lens for the target surface.

15. The optical imaging system according to claim 14, wherein the first step is greater than the second step.

16. The optical imaging system according to claim 14, wherein the controller is further configured to:
control, based on the first image focusing position, the driving module to drive the objective lens to move along the optical axis thereof at a third step, acquire, after each movement, a third position of the objective lens and control the image sensor to image the object of interest on the target surface to form a second image, and use the third position of the objective lens corresponding to the second image having the optimal image quality as a second image focusing position of the objective lens for the target surface; and
acquire a voltage generated by the focusing sensor based on the received first light beam reflected from the target surface when the objective lens is positioned at the second image focusing position and use the voltage as a focus tracking voltage.

17. The optical imaging system according to claim 16, wherein the controller is further configured to:
control, based on the second image focusing position, the driving module to drive the objective lens to move along the optical axis thereof at a fourth step under the focus tracking voltage; control, after each movement, the image sensor to image the object of interest on the target surface to form a third image; and compare the third image having the optimal image quality with the second image having the optimal image quality, wherein if the image qualities of the third image and the second image are substantially identical, it is determined that the focus tracking voltage passes verification.

18. The optical imaging system according to claim 16, wherein the controller is further configured to:
control the driving module to drive the objective lens to move to the second image focusing position under the focus tracking voltage; control the image sensor to image the objects of interest at a plurality of positions on the target surface; acquire a difference between the voltage generated by the focusing sensor based on the received first light beam reflected from the target surface and the focus tracking voltage in the process of imaging the objects of interest at each position by the image sensor; and control the driving module to drive the objective lens to move based on the difference to eliminate the difference.

19-26. (canceled)

27. A sequencing system, comprising the optical imaging system according to claim 14.

28. A computer-readable storage medium, wherein the medium has a program stored thereon, and the program is executable by a processor to implement the focusing method according to claim 1.
